(19) United States
(12) Patent Application Publication
(10) Pub. No.: US 2008/0274454 A1
(43) Pub. Date: Nov. 6, 2008

(54) Reversible and Chemically Programmable Micelle Assembly With Dna Block-Copolymer Amphiphiles

(76) Inventors: Chad A. Mirkin, Wilmette, IL (US); Zhi Li, Cambridge, MA (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/547,119

(22) PCT Filed: Apr. 6, 2005
(86) PCT No.: PCT/US05/11780

§ 371 Date: Aug. 28, 2007

Related U.S. Application Data
(60) Provisional application No. 60/560,833, filed on Apr. 7, 2004.

(51) Int. Cl. C12Q 1/68; C08G 81/02; C08L 53/00

(52) U.S. Cl. 435/6; 525/54.2

(57) ABSTRACT

The present invention is directed to amphiphilic block copolymers. More particularly the present invention is directed to amphiphilic block copolymers comprising a polynucleotide block and a hydrophobic polymer block, to micelles formed from the block copolymers, and to methods of using the micelles.

### CROSS-REFERENCE TO RELATED APPLICATION

This application claims the benefit of U.S. provisional application No. 60/560,833, filed Apr. 7, 2004.

### STATEMENT OF GOVERNMENTAL INTEREST

The government owns rights in the present invention pursuant to grant number DMR-0076097 from the National Science Foundation and grant number F 49620-02-0180 from the Air Force Office of Scientific Research.

### BACKGROUND

1. Field of the Invention

The present invention is directed to amphiphilic block copolymers. More particularly, the present invention is directed to block copolymers comprising a polynucleotide block and a hydrophobic polymer block, to micelles assembled from the block copolymers, and to methods of using these micelles in practical applications, such as phase transfer and recognition applications.

2. Background of the Invention

Amphiphilic block copolymers that contain at least one hydrophobic polymer block and one hydrophilic block generate ordered supramolecular structures, such as monolayers, micelles, vesicles, bilayers, helixes, and rod-and-sheet-like structures either in solution or at biphasic interfaces (Discher et al., Science, 297, 967-973, 2002; Discher et al., Curr. Opin. Colloid Interface Sci., 5, 125-131, 2000; Cornelissen, et al, Science, 293,676-680, 2001; Amphiphilic Block Copolymers: Self-assembly and Applications, Alexandridis, et al., eds., Elsevier: Amsterdam, 2000; Cornelissen, et al., Science, 280, 1427-1430, 1998; Shen, et al., Angew. Chem. Int. Ed. Eng., 39, 3310-3312, 2000; Zhang, et al., Science, 272, 1777-1779, 1996; Stupp, et al., Science, 276, 384-389, 1997). Applications for these polymeric amphiphiles include encapsulating agents for catalysts and drugs, surfactants for emulsions, adhesion promoters, and chemical separations.

Oligopeptides have been incorporated as blocks in such structures to provide scaffolding for assembly and subsequent chemical reactions within the larger supramolecular structures (Stupp, et al., Science, 276, 384-389, 1997; Hartgerink, et al., Science, 194, 1684-1687, 2001; Vauthey, et al., Proc. Natl. Acad. Sci. USA, 99, 5355-5360, 2002). Large polypeptide amphiphiles containing alternating polar and nonpolar regions have been investigated for their structural properties (Petka, et al., Science, 281, 389-392, 1998; Deming, et al., Nature, 417, 424-428, 2002). Micelle and vesicle structures have been formed from a “giant amphiphile” having a protein or enzyme as a hydrophilic group and a synthetic polymer as a hydrophobic group (Velonia, et al., J. Am. Soc. Chem., 124, 4224-4225, 2002; Boerakker, et al., Angew. Chem. Int. Ed. Eng., 41, 4239-4241, 2002).

Recently, significant research has focused on using DNA as a synthetically programmable interconnect for the preparation of materials having predetermined architectural parameters and properties (Storhoff, et al., Chem. Rev., 99, 1849-1862, 1999; Mirkin, et al., nature, 382, 607-609, 1996; Alivisatos, et al., Nature, 382, 609-611, 1996). Such materials have led to the development of biological detection schemes (Cao, et al., J. Am. Chem. Soc., 125, 14676-14677, 2003), nanostructures (Seeman, Science, 421, 427-431, 2003), and the construction of nanoelectronic devices (Keren, et al., Science, 302, 1380-1382, 2003). Researchers also focused on the construction of DNA-polymer hybrid materials which have been investigated for use in biodiagnostics and cellular uptake studies (Korri Youssoufi, et al., J. Am. Chem. Soc., 119, 7388-7389, 1997; Thompson, et al., J. Am. Chem. Soc., 125, 324-325, 2003; Watson, et al., J. Am. Chem. Soc., 123, 5592-5593, 2001; Jeong, et al., Bioconjugated Chem., 12, 917-923, 2001). Thus, production of molecules for use in biodiagnostics is beginning to gain significant attention in the theoretical context. There is a need to produce materials for use in biodiagnostic applications in medical and treatment protocols.

### SUMMARY OF THE INVENTION

The present invention relates to amphiphilic block copolymers comprising at least one hydrophilic polynucleotide block, such as DNA or RNA oligomers, and at least one hydrophobic polymer block, such as polystyrene. In particular, the present invention relates to an amphiphilic block copolymer having a general formula A-B, A-B-A, or B-A-B, wherein block A comprises a polynucleotide and block B comprises a hydrophobic polymer.

Therefore, one aspect of the present invention is to provide an amphiphilic block copolymer having a polynucleotide as a hydrophilic block. Additional arrangements of the A and B blocks, including a plurality of A and/or B blocks also are encompassed by the amphiphilic block copolymers of the invention. In another embodiment, a linking block, X, is positioned between one or more of the A-B linkages to provide a designed or predetermined spacing between the A and B blocks of the copolymer.

Another aspect of the invention is to provide supramolecular constructs comprising amphiphilic block copolymers of the present invention. In particular, the present invention provides these supramolecular constructs in the form of micelles. In certain embodiments, the supramolecular constructs are in the form of a sheet or tube.

Yet another aspect of the invention is use of micelles of the present invention in polynucleotide hybridization and recognition applications, as phase transfer agents, and as nanovesicles for delivery of compounds or compositions.

These and other aspects of the present invention will become apparent from the following detailed description of the preferred embodiments.

### BRIEF DESCRIPTION OF THE DRAWINGS

FIG. 1 is a gel electrophoretic migration-shift assay containing migration bands for an amphiphilic block copolymer (lanes 2 and 3) and for the starting polynucleotide (a DNA oligomer) (lane 1) in a 2% agarose gel;

FIG. 2 is an image generated from tapping mode atomic force microscopy (AFM) showing the spherical micelle structures constructed from an amphiphilic block copolymer of the invention; and

FIG. 3A is a schematic depicting the hybridization of a polynucleotide-polystyrene amphiphilic block copolymer micelle with a polynucleotide-gold nanoparticle and FIG. 3B is the corresponding melting curve of this hybridization.

### DETAILED DESCRIPTION OF THE PREFERRED EMBODIMENTS

The present invention relates to a polynucleotide-driven assembly of nanoparticles. More particularly, the present invention relates to amphiphilic block copolymers comprising a polynucleotide block and a hydrophobic polymer block. The amphiphilic block copolymers, formed using a solid phase synthesis strategy, are capable of assembling into a novel class of micelles. The assembled spherical micelles have recognition properties defined by the polynucleotide present in the amphiphilic block copolymer, and can be used to build higher-ordered structures through hybridization with materials that possess complementary polynucleotides. Additional structural shapes, such as sheets and tubes, also may be formed by present amphiphilic block copolymers having suitable numbers and arrangements of hydrophobic and hydrophilic blocks.

As used herein, the term “amphiphilic block copolymer” or “amphiphilic copolymer” refers to a compound comprising at least one polynucleotide block and at least one hydrophobic polymer block. Typically, the amphiphilic copolymer comprises one polynucleotide block and one hydrophobic polymer block.

As used herein, the term “polynucleotide” refers to an oligonucleotide or polymeric compound comprising bases of DNA, RNA, or combinations thereof. Alternatively, a polynucleotide is referred to herein as a “oligomer”, i.e., a DNA oligomer or RNA oligomer. Non-limiting examples of bases that comprise a polynucleotide used in the present invention include adenosine, guanosine, cytosine, thymidine, inosine, cytidine, uridine, pyrimidine, uracil, thymine, purine, methylcytosine, 5-hydroxymethylcytosine, 2-methyladenine, 1-methylguanine, 2,6-diaminopurine, 2-amino-6-chloropurine, 2,6-dichloropurine, 6-thioguanine, 6-iodopurine, 6-chloropurine, 8-azaadenine, allopurine, isoguanine, orotidine, xanthosine, xanthine, hypoxanthine, 1,2-diaminopurine, pseudouridine, C-5-propyne, isocytosine, isoguanine, 2-thiopyrimidine, rhodamines, benzimidazoies, ethidiums, propidiums, anthracyclines, mithramycins, acridines, actinomycins, merocyanines, coumarins, pyrenes, chrysenes, stilbenes, anthracenes, naphthalenes, salicylic acids, benzofurans, indodicarbocyanines, fluorescamine, psoralen, and other commercially available or synthesized bases.

Synthesized, or unnatural, bases or nucleosides can also be used in amphiphilic copolymers of the present invention. Such bases or nucleosides have an unnatural base structures, a sugar structure different from ribose or deoxyribose, or both. Examples of such synthesized bases or nucleosides include, but are not limited to, glycol based analogs (Zhang, et al, J. Am. Chem. Soc., 127, 417-44175, 2005), C-glycosides and base analogs (Kool, Acc. Curr. Res., 35, 936-943, 2002), peptide nucleic acids (Nielsen, et al., Acc. Chem. Res., 32, 624-630, 1999), and other sugar moiety analogs (Leumann, Bioorg. Med. Chem., 10, 841-854, 2002).

As used herein, a “sequence” refers to the order of bases in a polynucleotide or oligomer. For example, a DNA oligomer can have a sequence of 5′-AGCT-3′. A polynucleotide has a specific sequence from which its recognition properties are dependent.

One general synthetic scheme for preparing a novel amphiphilic copolymer of the present invention is depicted in Scheme I:

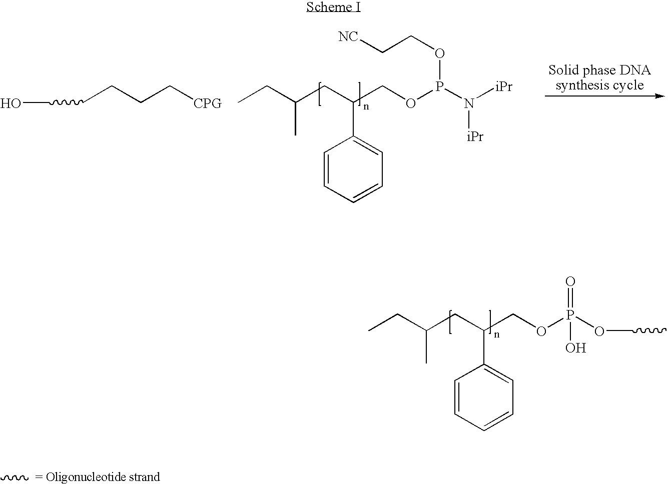

In this synthetic scheme, a DNA oligomer (i.e., a polynucleotide) is synthesized on a controlled pore glass support (CPG) using a DNA synthesizer. The polynucleotide then is reacted with a phosphoramidite derivative of a hydrophobic polymer (such as polystyrene) to form the desired amphiphilic block copolymer bound to the CPG solid support. The bound amphiphilic block copolymer then is cleaved from the solid support to provide the desired amphiphilic copolymer.

The amphiphilic block copolymers of the present invention are novel structures comprising a polynucleotide bound to a hydrophobic polymer, in the form of a block copolymer. Polynucleotides of varying lengths can be employed in the present invention. Typically, polynucleotides comprising about 5 to about 200 bases are present in the amphiphilic copolymers. Preferably, polynucleotides comprising about 5 to about 100 bases more preferably, about 5 to about 50 bases, and most preferably, about 5 to about 25 bases are present in the amphiphilic copolymers of the present invention. The oligonucleotides of 15, 20, 25, 30, 35, 40, 45, 50, 55, 60, 65, 70, 75 or more bases in length are specifically contemplated. Any given micelle or hybridizing structure may comprise a plurality of individual oligonucleotides. Moreover, the plurality of individual oligonucleotides may each be of uniform length or may be of varying length.

The hydrophobic polymer block present in a amphiphilic copolymer is not limited. The hydrophobic block is an uncrosslinked polymer. The identity of the hydrophobic polymer, and its molecular weight, are judiciously selected, together with the identity of the polynucleotide, to provide the desired or predetermined properties of the amphiphilic block copolymer. Such a selection of a polynucleotide and a hydrophobic polymer is well within the skill of persons practicing in the art.

The hydrophobic polymer block of the amphiphilic copolymer can be a homopolymer or a copolymer. The hydrophobic polymer block is an uncrosslinked polymer. Hydrophobic polymers useful in the present invention include, but are not limited to, a block of polystyrene, polyethylene, polybutylene, polypropylene, polymerized mixed olefins, polyterpene, polyisoprene, polyvinyltoluene, poly(α-methylstyrene), poly(o-methylstyrene), poly(m-methylstyrene), poly(p-methylstyrene), poly(dimethylphenylene oxide), polyurethane, polyvinyl chloride, polyimide, polyvinylacetate, and mixtures thereof.

The hydrophobic polymer block also can comprise copolymers prepared from monomers utilized in the above list of homopolymers. Such copolymers include, but are not limited to, poly(butadiene-co-styrene), poly(ethylene-co-propylene), poly(ethylene-co-propylene-co-5-ethylidene-2-norborene), poly(butadiene-co-acrylonitrile), poly(isobutylene-co-isoprene), poly(vinyl chloride-co-vinylidene chloride), poly(styrene-co-acrylonitrile), and mixtures thereof. The hydrophobic polymer block can be a random copolymer or can be a block copolymer itself comprising discrete domains of different homopolymers. Nonlimiting examples of such a block copolymer include alternating blocks of any of the aforementioned homopolymers, i.e., polybutylene and polystyrene, polyethylene and polyisoprene, and polyethylene and polypropylene.

As used herein, a “hydrophobic polymer” is a polymer which is insoluble, only slightly soluble, or does not form a stable dispersion in water. Typically, a polymer having a solubility or dispersibility in water of less than about 0.1 g/100 mL at 25° C. is hydrophobic.

The hydrophobic polymer used in the formation of an amphiphilic block copolymer can have a wide range of molecular weights. Typically, the hydrophobic polymer has a molecular weight of about 1 to about 100 kDa. However, the hydrophobic polymer can have a molecular weight of less than about 1 kDa or more than about 100 kDa. The molecular weight of the hydrophobic polymer is determined after a consideration of the desired properties of the amphiphilic copolymer, the micelles prepared from the amphiphilic copolymer, and the end use application of the amphiphilic copolymer or micelles prepared therefrom.

In preferred embodiments, the molecular weight of the hydrophobic polymer is about 2 to about 50 kDa, more preferably, about 3 to about 30 kDa, and most preferably, about 4 to about 10 kDa. Specific molecular weights include about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, and about 20 kDa.

Amphiphilic block copolymers of the present invention also can contain an optional linking polymeric block. The linking block links the hydrophobic polymer block to the hydrophilic polynucleotide block of a present amphiphilic copolymer, and serves to spatially separate the hydrophobic and hydrophilic blocks of the present copolymer. The linking block typically comprises one or more monomers such that the linking block has both hydrophobic and hydrophilic attributes, but the overall properties of the linking block are neither hydrophobic nor hydrophilic. Examples of monomers useful in the formation of the linking block include, but are not limited to, styrene, ethylene, butylene, propylene, mixed olefins, terpene, isoprene, vinyl toluene, α-methylstyrene, o-methylstyrene, m-methylstyrene, p-methylstyrene, dimethylphenylene oxide, urethane, vinyl chloride, imides, vinylacetate, acrylic acid, methacrylic acid, acrylonitrile, vinyl alcohol, ethylene glycol, propylene glycol, butylene glycol, maleic anhydride, acrylamide, methacrylamide, a C₁₋₄ alkyl acrylate, a C₁₋₆ alkyl methacrylate, phthalic anhydride, terephthalic acid, isophthalic acid, succinic anhydride, and mixtures thereof.

The linking block of an amphiphilic block copolymer can have a wide range of molecular weights. Typically, the linking block has a molecular weight of about 0.5 to about 10 kDa. However, the linking block can have a molecular weight of less than about 0.5 kDa or more than about 10 kDa. The molecular weight of the linking block is determined after a consideration of the desired properties of the amphiphilic copolymer, the micelles prepared from the amphiphilic copolymer, and the end use application of the amphiphilic copolymer or micelles prepared therefrom.

In preferred embodiments, the molecular weight of the linking block is about 0.5 to about 9 kDa, more preferably, about 0.5 to about 8 kDa and most preferably, about 0.5 to about 7 kDa. Specific molecular weights include about 0.5 kDa, about 1 kDa, about 2 kDa, about 3 kDa, about 4 kDa, about 5 kDa, about 6 kDa, about 7 kDa about 8 kDa, about 9 kDa, and about 10 kDa.

Amphiphilic copolymer of the present invention that contain a linking block can be prepared as set forth in Scheme I. The linking block first can be bound to the polynucleotide block, following by binding to the hydrophobic polymer block. Preferably, the linking block is bound to the hydrophobic polymer block, followed by binding to the polynucleotide block.

The amphiphilic copolymer of the present invention therefore comprises (a) at least one hydrophilic polynucleotide block, i.e., at least one block A, (b) at least one hydrophobic polymer block, i.e., at least one block B, and (c) one or more optional linking block, i.e., one or more optional linking block X. The amphiphilic copolymers generally have a general structural formula A-B, A-B-A, or B-A-B. The amphiphilic copolymer also can contain a plurality of A-B arrangements, i.e., (A-B)ₙ, wherein n is an integer of 1 to 10, preferably 1 to 5. In embodiments wherein an optional linking block is present, the amphiphilic copolymer has a general structural formula (A-X-B)ₙ. A present amphiphilic copolymer can be terminated with one A block and one B block, two A blocks, or two B blocks.

The amphiphilic block copolymers of the present invention form micelles having unique properties when admixed with a solvent. Micelles prepared from the amphiphilic block copolymers can form stable suspensions in a variety of polar and nonpolar solvents, for example methylene chloride, tetrahydrofuran, dimethylformamide, and water. This is an important property because polynucleotides by themselves are essentially insoluble in methylene chloride, tetrahydrofuran, and, other nonpolar solvents; and hydrophobic polymers are essentially insoluble in water and other polar solvents, such as alcohols and diols.

When an amphiphilic block copolymer of the present invention is admixed with a polar solvent, a micellular supramolecular construct forms. In this embodiment, the hydrophilic portion of the amphiphilic block copolymer (i.e., the polynucleotide) forms the outer shell of the micelle, and the hydrophobic polymer block of the amphiphilic block copolymer is in the center of the micelle, directed away from the incompatible polar solvent (e.g., water). When an amphiphilic block copolymer is admixed with a nonpolar solvent (e.g., methylene chloride), the resulting micelles have the hydrophobic block of the amphiphilic copolymer on the outer surface of the micelle, and the hydrophilic block of the amphiphilic copolymer is in the center of the micelle, directed away from the incompatible nonpolar solvent.

The size of the micelles is directly related to the identity and size of the amphiphilic block copolymers used to form the micelles. Typical diameters of micelles formed from amphiphilic copolymers of the present invention range from about 3 nm to about 500 nm. However, the present micelles can have diameters that are less than about 3 nm and greater than about 500 nm. The diameter of the present micelle is determined by the properties of the amphiphilic copolymer from which the micelle is formed.

The particular identity and size of the hydrophobic, hydrophilic, and linking blocks of an amphiphilic copolymer provide additional structures. For example, a sheet or tubular structure can be formed by an arrangement wherein hydrophilic blocks are the capping, or terminal, segments of the amphiphilic copolymer, with variously arranged hydrophobic blocks, and, optionally, linking blocks.

In preferred embodiments, a present micelle has a diameter of about 3 nm to about 500 nm, more preferably, about 5 nm to about 100 nm, and most preferably, about 8 nm to about 50 nm. For example, micelles having an average diameter of about 8 to about 30 nm can be formed by preparing an amphiphilic block copolymer from polynucleotides containing 5 bases, 10 bases, or 25 bases and a polystyrene having a molecular weight of 4.1 kDa, 7.2 kDa or 9.5 kDa. Spherical micelles typically are formed, although cylindrical rod structures also may be formed in small amounts.

When an amphiphilic block copolymer is admixed with a polar solvent, a micelle forms and the polynucleotide of the amphiphilic copolymer is exposed to the solvent. Advantageously, these micelles have an ability to hybridize with a hybridizing structure that contains a complementary polynucleotide sequence, similar to the recognition properties of a polynucleotide sequence that is free of bonding to a hydrophobic polymer. Therefore, another embodiment of the present invention is a method of recognizing a hybridizing structure that includes a complementary polynucleotide sequence to the polynucleotide sequence present in micelles of an amphiphilic block copolymer.

For example, a micelle formed from an amphiphilic block copolymer comprising a polynucleotide and polystyrene can recognize a hybridizing structure that includes a polynucleotide sequence that is complementary to the polynucleotide sequence present in the amphiphilic copolymer. The hybridizing structure can be either a polynucleotide sequence itself or a polynucleotide bound to a support structure, such as, for example, a metal (e.g., gold, silver, titanium, or nickel) nanoparticle, a protein, a polypeptide, a hydrophobic polymer, a solid support, an antibody, a fluorophore, a magnetic bead, a dye, a catalyst, a ligand for a metal, a ligand complexed to a metal, or a saccharide or polysaccharide.

As used herein, a “hybridizing structure” refers to a compound or entity comprising a complementary polynucleotide that is capable of selectively and specifically hydrogen bonding to the polynucleotide present in an amphiphilic block copolymer of the invention. The polynucleotide of the hybridizing structure can be completely complementary to the polynucleotide of the amphiphilic block copolymer or can contain one or more base mismatches with the polynucleotide of the amphiphilic block copolymer.

The recognition properties of the polynucleotide of the micelle can be used in detection, identification, and assaying techniques currently known in the art. See, for example, U.S. Patent Publication Nos. 2004/0219533, 2005/0026181, and 2003/0096113, and PCT Publication No. WO 2005/003394, each of which is incorporated by reference.

Micelles formed from the amphiphilic block copolymers of the invention also can be used as phase transfer agents, in general, or as nanovesicles, in particular. As a phase transfer agent, the micelle can be used to transfer or shepherd a compound or composition from a phase in which the compound or composition is soluble to a second phase in which it is insoluble, or vice versa. Nanovesicles perform similarly to phase transfer agents, but are used in cellular transport applications to shepherd compounds across a cell membrane, for example.

Because of this shepherding effect, the present micelles have a wide range of applications, such as in dual phase catalytic reactions, in cell transport, and in other applications wherein more than one phase of a multiphase system is of interest. Phase transfer agents are useful in control of reaction rate, extraction of product, longevity of catalytic reaction, and in separation of reaction components.

An important property of the micelles formed from the present amphiphilic block copolymers is reversibility of the micellular superstructure formation. Therefore, a present micelle can serve its intended purpose in a system (i.e., as a transport agent or for recognition purposes), then the micelle can be dispersed into the original, unstructured amphiphilic block copolymer by changing the physical characteristics of the system, e.g., a change from a polar to a nonpolar system or solvent. The solubility properties of the amphiphilic copolymer, and the solvent in which the amphiphilic copolymer is dispersed, dictate the structure of the micelles that form, and manipulation of these solubility properties and identity of the solvent allow for both micelle formation and dispersion. For example, micelles can be used to recognize and hybridize with a complementary polynucleotide sequence as a means of identifying or purifying that complementary polynucleotide. After the complementary polynucleotide has been identified or purified, the micelles can be dispersed, and the complementary polynucleotide of interest can be isolated from the amphiphilic copolymer by dehybridization.

Another important property of the micelles formed from the present amphiphilic block copolymers is their ability to hybridize with complementary sequences of a hybridizing structure that is capable of detecting a marker of a biological sample. Therefore, another embodiment of the present invention is a method of detecting a marker in a biological sample comprising the steps of contacting the biological sample with a hybridizing structure that comprises a first polynucleotide that can hybridize to a marker in the biological sample and a second polynucleotide that can hybridize to a polynucleotide of a micelle of the present invention, under conditions that allow hybridization of the polynucleotides of the hybridizing structure, micelle, and marker in the biological system, and then detecting the hybridization that occurs.

According to certain aspects of the present invention, the hybridizing structures of the invention are used to detect markers in a biological sample. These hybridizing structures comprise oligonucleotides of at least 17, at least 18, at least 19, at least 20, at least 22, at least 25, at least 30 or at least 40, bases that specifically hybridize with the isolated nucleic acid molecules from biological samples. As described herein, the hybridizing structures also contain oligonucleotides that hybridize with complementary oligonucleotides on the present micelles. A sequence is “specifically homologous” to another sequence if it specifically hybridizes to the a complement of itself. The complement of the sequence may be an exact complement or may be mismatched. Those of skill in the art are aware of conditions under which sequences that are less than completely complementary will nonetheless hybridize with each other. A sequence “specifically hybridizes” to another sequence if it hybridizes to form Watson-Crick or Hoogsteen base pairs either in the body, or under conditions which approximate physiological conditions with respect to ionic strength, e.g., 140 mM NaCl, 5 mM MgCl₂. Hybridization of oligonucleotides from the hybridizing structure to the micelles or to nucleic acids isolated from biological samples will employ similar techniques. It is known that hybridization of shorter polynucleotides (below 200 bases in length, e.g. 17-40 bases in length) can be performed at high stringency, moderate stringency or mild (or low) stringency hybridization conditions.

Exemplary high stringency hybridization is performed using a hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, with a final wash solution of 3 M TMACI, 0.01 M sodium phosphate (pH 6.8), 1 mM EDTA (pH 7.6), 0.5% SDS at 1-1.5° C. below the Tₘ; moderate stringency hybridization is performed using a hybridization buffer solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, with a final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; whereas mild hybridization is performed using a hybridization solution of 6×SSC and 1% SDS or 3 M TMACI, 0.01 M sodium phosphate (pH16.8), 1 mM EDTA (pH 7.6), 0.5% SDS, 100 μg/ml denatured salmon sperm DNA and 0.1% nonfat dried milk, hybridization temperature of 37° C., with a final wash solution of 6×SSC and final wash at 22° C.

Those skilled in the art understand that the hybridization temperature is important for the hybridization conditions and that the less stringent the hybridization conditions, the less specific the hybridization. Hybridizations carried out at 55° C. are considered to be at low stringency, more preferable and specific hybridizations occur at medium stringency conditions which typically employ temperatures of at least 60° C., still more preferable and specific hybridization occur at temperatures of at least 65° C. which are considered medium/high stringency conditions. Hybridizations carried out at temperature of about 70° C.-75° C. are considered high to very high stringency.

Methods and compositions for performing nucleic acid hybridization are well known to those of skill in the art and are described in e.g., Sambrook et al., MOLECULAR CLONING: A LABORATORY MANUAL, 3d ed., 2001. The hybridization may be under low stringency conditions, medium stringency conditions, high stringency conditions or very high stringency conditions.

Detection of the hybridization can occur through a variety of means. If desired, the hybridizing structure may be labeled, for instance, with biotin, a radiolabel, or fluorescent label. Suitable fluorescent labels are known in the art and commercially available from, for example, Molecular Probes (Eugene, Oreg.). These include, e.g., donor/acceptor (i.e., first and second signaling moieties) molecules such as: fluorescein isothiocyanate (FITC)/tetramethylrhodamine isothiocyanate (TRITC), FITC/Texas Red™ Molecular Probes), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X (ROX), FITC/tetramethylrhodamine (TAMRA), and others. In addition to the organic fluorophores already mentioned, various types of nonorganic fluorescent labels are known in the art and are commercially available from, for example, Quantum Dot Corporation, Inc. (Hayward Calif.). These include, e.g., donor/acceptor (i.e., first and second signaling moieties) semiconductor nanocrystals (i.e., “quantum dots”) whose absorption and emission spectra can be precisely controlled through the selection of nanoparticle material, size, and composition (see, for example, Bruchez et al., Science, 281, 2013-2015, 1998, Chan et al, Science, 281, 2016-2018, 1998; Brenner et al., Nature Biotech., 19, 630-634, 2001). Any other detection method can also be used in the detection and/or quantification of targets. For example, radioactive labels could be used, including ³²P, ³³P, ¹⁴C, ³H, or ¹²⁵I. Also enzymatic labels can be used including horse radish peroxidase or alkaline phosphatase. The detection method could also involve the use of a capture tag for the bound nucleic acid sensor molecule. Quantitation of the captured fluorescence, radio, or other signal provides a means for inferring the concentration of marker molecule in the biological sample.

A wide variety of markers or genes in a biological sample may be detected using the compositions described herein. Such markers include, for example, genes that encode immunoglobulins, cytokines, enzymes, hormones, cancer antigens, nutritional markers, tissue specific antigens, markers for autoimmune diseases, etc. The types of markers of interest in the present invention are specifically disclosed in U.S. Pat. No. 4,650,770, the disclosure of which is incorporated by reference herein in its entirety.

The biological sample may be obtained from an animal and any be any biological sample typically employed in diagnostic assays. For example, biological samples may be from a fluid such as urine, blood, plasma, serum, saliva, semen, stool, sputum, cerebral spinal fluid, tears, mucus, and the like.

Other embodiments of this invention contemplate kits that comprise the individual components for the preparation of the compositions and performing the methods of the invention described herein. A kit according to the invention may comprise, e.g., the micelles, hybridizing structures, suitable buffer solutions and/or other reagents necessary to perform methods of the invention. An exemplary kit is one which comprises a first composition comprising micelles described herein; a second composition comprising the hybridizing structures described herein and instructions for performing a diagnostic assay. Preferably, such kits are used in diagnostic methods and comprise the relevant substrates and materials needed for the collection of biological samples from a subject.

In addition, the kits may comprise one or more enzymes for the PCR amplification and/or for the reverse transcription reactions for use in isolating nucleic acids from a biological sample. Thus, the kits optionally also includes one or more of: a polymerase (e.g., a polymerase having or substantially lacking 5′ to 3′ nuclease activity), a buffer, a standard template for calibrating a detection reaction, instructions for extending the primers to amplify at least a portion of the target nucleic acid sequence or reverse complement thereof, instructions for using the components to amplify, detect and/or quantitate the target nucleotide sequence or reverse complement thereof, or packaging materials. The kits may also preferably include the deoxyribonucleoside triphosphates (typically dATP, dCTP, dGTP, and dTTP, although these can be replaced and/or supplemented with other dNTPs, e.g., a dNTP comprising a base analog that Watson-Crick base pairs like one of the conventional bases, e.g., uracil inosine, or 7-deazaguanine), an aqueous buffer, and appropriate salts and metal cations (e.g., Mg²⁺).

The kit may comprise a solid support on which the present the biological samples being tested. The solid support may be any support that is typically used in nucleic acid preparation and analysis. Such supports include, but are not limited to plastic, glass, beads, microtiter plates. Indeed, glass, plastics, metals and the like are often used, and the nucleic acid amplification method of the present invention can be used irrespective of the type of the substrate.

In addition to the above, the kits may comprise components as standards. For example, the kits may comprise a known nucleic acid sequences such that the signal received from the environmental/biological sample can be compared with that received from the standard to ensure the integrity of the assay components and conditions.

### EXAMPLES

A method of preparing and using amphiphilic block copolymers of the present invention is set forth below. In the following examples, a DNA oligomer-polystyrene block copolymer is disclosed for illustrative purposes, and is used to exemplify the procedure as well as the micellular properties. The following examples are not intended to limit the scope of the compounds, compositions, or methods described herein.

### Example 1

A DNA oligomer (i.e., 5′-ATCCTTATCAATATT-3′) attached to a CPG support was produced using a DNA synthesizer and standard coupling techniques (coupling protocols vary according to the synthesizer used). The oligomer was terminated with a 5′ hydroxyl group. See Scheme I. A phosphoramidite coupled polystyrene for binding to the DNA oligomer was synthesized as shown in Scheme II:

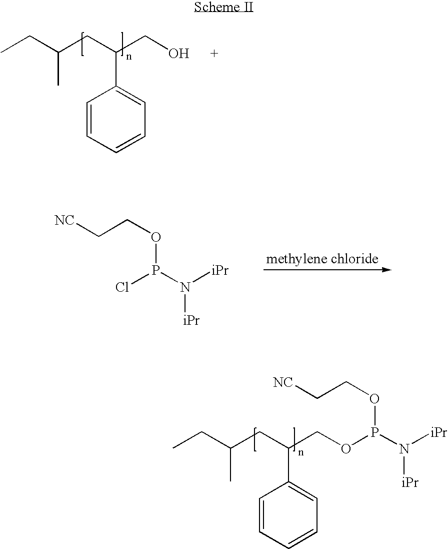

More particularly, a hydroxylated polystyrene (M_{n, avg}=5.6×10³) (van Hest, et al. Chem. Eur., 2, 1616-1626, 1996) was reacted with chlorophosphoramidite in anhydrous methylene chloride, followed by precipitation in acetonitrile, to provide the polystyrene phosphoramidite as a mixture of diasteromers (³¹P NMR 148.7 ppm and 148.2 ppm).

The supported DNA oligomer then was coupled to the polystyrene phosphoramidite using a syringe-synthesis technique (Storhoff, et al., J. Am. Chem. Soc., 120, 1959-1964, 1998). After a 3 hour reaction time, excess phosphoramidite was removed by rinsing the CPG-bound copolymer with methylene chloride, then dimethylformamide. The amphiphilic block copolymer then was deprotected and cleaved from the CPG solid support using ammonium hydroxide. The resulting amphiphilic block copolymer was dissolved in dimethylformamide to determine the concentration of the amphiphilic copolymer. The solution was measured for concentration of amphiphilic block copolymer using standard DNA detection techniques. Typically, 300 to 6000D (optical density at 269 nm in dimethylformamide) of the amphiphilic block copolymer product was collected. The molecular weight and structural assignment were confirmed using MALDI-TOF mass spectrometry (M_{n, avg}=11.7×10³, trans-3-indoleacrylic acid as matrix).

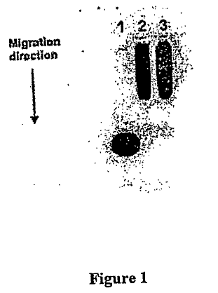

The purity of the amphiphilic block copolymer was assessed using a gel electrophoretic migration shift assay. FIG. 1 is a gel-shift assay in which lane 1 shows the migration of the DNA oligomer prior to appending the hydrophobic polymer. Lanes 2 and 3 show the migration of the amphiphilic block copolymer formed after the reaction outlined in Scheme I. It is noted that no DNA oligomer remains after the reaction, and that the resulting copolymer has a slower mobility on the 2% agarose gel than the starting DNA oligomer. This slower mobility indicates that a higher molecular weight entity has been prepared. The amphiphilic block copolymer (lanes 2 and 3) moves along the migration direction significantly slower than its DNA component because of the covalently attached polymer block and the existence of assembled structures. Overall, the gel-shift assay indicates the formation of the desired block copolymer containing the DNA oligomer and the hydrophobic polystyrene.

### Example 2

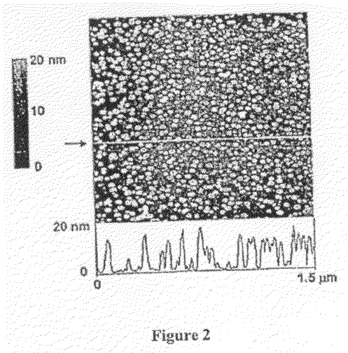

The amphiphilic block copolymers of the present invention form stable suspensions in a variety of solvents, including methylene chloride, dimethylformamide, tetrahydrofuran, and water. To assess the type of structures formed from these novel amphiphilic block copolymers, 35 OD solution of the amphiphilic block copolymer formed in Example 1 in dimethylformamide (1 mL) was gradually diluted with 9 mL water. The majority of the dimethylformamide then was removed from the mixture by dialysis. After dialysis, the resulting solution was allowed to incubate at room temperature for 24 hours. Centrifugation of 5000 revolutions per minute (rpm) for 10 minutes (min) then was used to remove heavily aggregated structures from the cloudy solution. The resulting clear solution contained micelles of the amphiphilic block copolymer. The micellular structure was confirmed using tapping mode atomic force microscopy (AFM), which revealed a dense layer of spherical particles having diameters between 13 and 18 nm. In particular, FIG. 2 is a tapping mode atomic force microscopy (AFM) spectrum that illustrates the spherical micelles formed from an aqueous dispersion of the DNA oligomer-polystyrene amphiphilic block copolymer synthesized in Example 1. To obtain this image, a drop of micelle solution (5 μL) was placed on an aminopropyltrimethoxysilane functionalized mica surface, which then was sprayed with dry nitrogen, washed with deionized water, and dried again with flowing nitrogen, before the AFM image was obtained.

The size distribution of micelles formed from the amphiphilic block copolymer of Example 1 also was measured in solution via dynamic light scattering, which showed an average particle diameter of 16.4 nm (25% polydispersity, quadratic simulation). This result is consistent with the measurements from the tapping mode AFM.

To assess the affect of amphiphilic block copolymer size and identity on the diameter of the resulting micelles, a series of amphiphilic block copolymers, which varied in DNA oligomer length (5 bases, 10 bases, and 25 bases) and polystyrene molecular weight (4.1 kDa, 7.2 kDa, and 9.5 kDa), were synthesized using the protocol outlined in Example 1. The diameters of the nine resulting micelle compositions varied from 8 nm to 30 nm n.

### Example 3

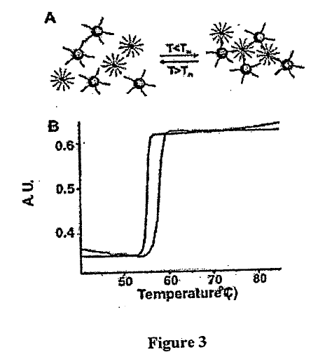

The recognition properties of micelles formed from a present amphiphilic copolymer were assessed using known DNA hybridization experimental techniques. Micelles of the amphiphilic copolymer of Example 1 were formed in water, such that the hydrophilic DNA oligomers formed the outer sphere of the micelles, thereby leaving the DNA oligomers accessible to the surrounding solution environment and permitting hybridization or recognition of a species in solution having a complementary polynucleotide sequence. A solution containing the micelles formed from the amphiphilic block copolymer of Example 1 was treated with a solution of 13 nm gold nanoparticles modified with a complementary DNA sequence (i.e., 3′-TAGGAATAGTTATAA-A₅-SH-5′) in 0.3M sodium chloride and 10 mM phosphate buffer solution (see Mirkin, et al., Nature, 382, 607-609, 1996 and Alivisatos, et al., Nature, 382, 609-611, 1996). The aggregates formed through hybridization (FIG. 3A) were monitored through the surface plasmon band of the gold nanoparticles at 520 nm. Because hybridization is a temperature dependent process, alteration of solution temperature affects the hybridization between the hybridizing structure and the micelles of the amphiphilic copolymer. The results of a melting experiment of this system are shown in FIG. 3B, right-most curve. The sharp melting transition denotes the disassembly of the aggregates and indicates that the DNA oligomer of the amphiphilic copolymer and this DNA sequence of the modified gold nanoparticle are no longer hybridized (Tₘ=57.8° C.). FIG. 3A)

A hybridization process is highly sequence specific. A 13 nm gold nanoparticle modified with a DNA sequence containing one base mismatch (i.e. 3′-TAGGAATATTTATAA-A₅-SH-5′) to that of the DNA-polystyrene amphiphilic block copolymer of Example 1 showed a lower melting temperature (Tₘ=55.2° C.) as a result of the incomplete hybridization between the micelle and gold nanoparticle. See FIG. 3B, left-most curve. The origin of these sharp melting curves can be explained by a cooperative melting model as described in Watson, et al., J. Am. Chem. Soc., 123, 5592-5593, 2001 and Jin, et al., J. Am. Chem. Soc., 125, 1643-1654, 2003.

What is claimed is:

1. An amphiphilic block copolymer comprising at least one hydrophobic block and at least one hydrophilic block, said hydrophilic block comprising a polynucleotide.

2. The amphiphilic block copolymer of claim 1 having a general formula (A-B)ₙ, wherein A is a hydrophilic block comprising a polynucleotide, B is a hydrophobic block comprising a hydrophobic polymer, and n is an integer of 1 to 10.

3. The amphiphilic block copolymer of claim 1 having a general formula A-B-A or B-A-B, wherein A is a hydrophilic block comprising a polynucleotide and B is a hydrophobic block comprising a hydrophobic polymer.

4. The amphiphilic block copolymer of claim 1 having a general formula (A-X-B)ₙ wherein A is a hydrophilic block comprising a polynucleotide, B is a hydrophobic block comprising a hydrophobic polymer, X is a linking polymer block, and n is an integer of 1 to 10.

5. The amphiphilic block copolymer of claim 2, 3, or 4 wherein the polynucleotide block A is selected from the group consisting of a DNA oligomer, a RNA oligomer, and mixtures thereof.

6. The amphiphilic block copolymer of claim 2, 3, or 4 wherein the polynucleotide block A comprises about 5 to about 200 bases.

7. The amphiphilic block copolymer of claim 6 wherein the polynucleotide block A comprises about 5 to about 100 bases.

8. The amphiphilic block copolymer of claim 7 wherein the polynucleotide block A comprises about 5 to about 25 bases.

9. The amphiphilic block copolymer of claim 2, 3, or 4 wherein the hydrophobic polymer block B has a molecular weight of about 1 to about 100 kDa.

10. The amphiphilic block copolymer of claim 9 wherein the hydrophobic polymer block B has a molecular weight of about 2 to about 50 kDa.

11. The amphiphilic block copolymer of claim 10 wherein the hydrophobic polymer block B has a molecular weight of about 4 to about 25 kDa.

12. The amphiphilic block copolymer of claim 2, 3, or 4, wherein the hydrophobic polymer block B is a homopolymer.

13. The amphiphilic block copolymer of claim 2, 3, or 4 wherein the hydrophobic polymer block B is selected from the group consisting of polystyrene, polyethylene, polybutylene, polypropylene, polymerized mixed olefins, polyterpene, polyisoprene, polyvinyltoluene, poly(α-methylstyrene), poly(o-methylstyrene), poly(m-methylstyrene), poly(p-methylstyrene), poly(dimethylphenylene oxide), polyurethane, polyvinyl chloride, polyimide, polyvinylacetate, and mixtures thereof.

14. The amphiphilic block copolymer of claim 2, 3, or 4 wherein the hydrophobic polymer block B comprises polystyrene.

15. The amphiphilic block copolymer of claim 2, 3, or 4 wherein the hydrophobic polymer block B is a copolymer.

16. The amphiphilic block copolymer of claim 4 wherein the linking block X has a molecular weight of about 0.5 to about 10 kDa.

17. The amphiphilic block copolymer of claim 4 wherein the linking block X is a homopolymer or a copolymer comprising one or more monomers selected from the group consisting of styrene, ethylene, butylene, propylene, mixed olefins, terpene, isoprene, vinyl toluene, α-methylstyrene, o-methylstyrene, m-methylstyrene, p-methylstyrene, dimethylphenylene oxide, urethane, vinyl chloride, imides, vinylacetate, acrylic acid, methacrylic acid, acrylonitrile, vinyl alcohol, ethylene glycol, propylene glycol, butylene glycol, maleic anhydride, acrylamide, methacrylamide, a C₁₋₆ alkyl acrylate, a C₁₋₆ alkyl methacrylate, phthalic anhydride, terephthalic acid, isophthalic acid, succinic anhydride, and mixtures thereof.

18. A supramolecular construct comprising the amphiphilic block copolymers of claim 1.

19. The supramolecular construct of claim 18 in the form of a micelle, a sheet, or a tube.

20. The micelle of claim 19 having a spherical shape.

21. The micelle of claim 19 having an average diameter of about 3 to about 500 nm.

22. The micelle of claim 21 having an average diameter of about 5 to about 100 nm.

23. The micelle of claim 22 having an average diameter of about 8 to about 50 nm.

24. The micelle of claim 19 wherein the micelle is formed in a polar solvent.

25. The micelle of claim 19 wherein the micelle is formed in a nonpolar solvent.

26. A composition comprising
a) a micelle comprising amphiphilic block copolymers of claim 1, and
b) a hybridizing structure comprising a polynucleotide, wherein the micelle and the hybridizing structure hybridize through hybridization of the hydrophilic polynucleotide block of the amphiphilic block copolymer and the polynucleotide of the hybridizing structure.

27. The composition of claim 26 wherein the polynucleotide of the hybridizing structure is complementary to the polynucleotide of the hydrophilic block

28. The composition of claim 26 wherein the polynucleotide of the hybridizing structure contains at least one base mismatch with the polynucleotide of the hydrophilic block.

29. The composition of claim 26 wherein said hybridizing structure further comprises a metal nanoparticle.

30. The composition of claim 29 wherein the metal nanoparticle comprises a metal selected from the group consisting of gold, silver, nickel, and titanium.

31. The composition of claim 26 wherein said hybridizing structure further comprises a detectable label.

32. The composition of claim 31 wherein the detectable label is selected from the group consisting of a fluorescent label or a radiolabel.

33. The composition of claim 26 wherein said hybridizing structure further comprises a polynucleotide that hybridizes with a marker in a biological system.

34. A method of detecting the presence of a marker in a biological sample comprising contacting said sample with a composition of claim 33 under conditions that allow hybridization of said hybridizing structure to said marker in said biological sample.

35. A method of detecting the presence of a marker in a biological sample comprising the steps of:
a) contacting the biological sample with:
i) a hybridizing structure that comprises a first polynucleotide that hybridizes to a marker in said biological sample and a second polynucleotide that hybridizes to a polynucleotide located on a micelle under conditions that allow hybridization of said hybridizing structure to said marker in said biological sample, and
ii) a micelle comprising an amphiphilic block polymer having a general structure A-B, A-B-A, or B-A-B, wherein A is a hydrophilic block comprising a polynucleotide that will hybridize to a complementary nucleic acid on the hybridizing structure of step (a) and B is a hydrophobic block comprising a hydrophobic polymer, under conditions that allow hybridization of said micelle structure to said hybridizing structure, and
b) detecting the hybridization of step (a) with said biological sample.

36. The method of claim 35 wherein said micelle is contacted with said hybridizing structure prior to contacting with said biological sample.

37. The method of claim 35, wherein said micelle is contacted with said hybridizing structure after said hybridizing structure has been hybridized with said biological sample.

38. The method of claim 35 wherein said marker is a marker of a biological disorder.

39. The method of claim 38 wherein said biological disorder is a cancer or an autoimmune disease.

40. The methods of claim 35 wherein said hybridization structure comprises polynucleotides that individually hybridize to a plurality of genes in said biological sample.

41. A kit comprising
a) an aqueous solution of micelles of amphiphilic block copolymers of claim 1, 2, 3, or 4;
b) a solution of buffer for forming the necessary salt conditions for hybridization of the polynucleotide of the amphiphilic block copolymer to a complementary polynucleotide sequence.

42. A kit of claim 41 further comprising a composition comprising a hybridizing structure that comprises a first polynucleotide that hybridizes to a biological marker and a second polynucleotide that hybridizes to a polynucleotide located on the micelle of (a).


SEQUENCE LISTING (3 sequences)

SEQ ID NO: 1 - DNA, 15 bp, Artificial sequence
  Synthetic primer
atccttatcaatatt

SEQ ID NO: 2 - DNA, 20 bp, Artificial sequence
  Synthetic primer
naaaaaatattgataaggat

SEQ ID NO: 3 - DNA, 20 bp, Artificial sequence
  Synthetic primer
naaaaaatatttataaggat